(19) United States
(12) Patent Application Publication
(10) Pub. No.: US 2022/0071595 A1
(43) Pub. Date: Mar. 10, 2022

(54) METHOD AND SYSTEM FOR ADAPTING USER INTERFACE ELEMENTS BASED ON REAL-TIME ANATOMICAL STRUCTURE RECOGNITION IN ACQUIRED ULTRASOUND IMAGE VIEWS

(71) Applicant: GE Precision Healthcare LLC, Wauwatosa, WI (US)

(72) Inventors: Martin Paul Mienkina, Neumarkt am Wallersee (AT); Andrew Lewis Barker, Pöndorf, (AT); Andreas Haas, Schoerfling (AT); Bálint Czupi, Seewalchen am Attersee (AT)

(21) Appl. No.: 17/016,623

(22) Filed: Sep. 10, 2020

(51) Int. Cl. A61B 8/00; G06F 3/0481; G06F 3/0482; G06T 11/60; G06F 3/0484

(52) U.S. Cl. CPC A61B 8/463; G06F 3/04815; G06F 3/0482; A61B 8/467; G06F 3/0484; A61B 8/466; G06T 11/60

(57) ABSTRACT

A system and method for adapting user interface elements based on real-time anatomical structure recognition in acquired ultrasound image views is provided. The method includes acquiring, by an ultrasound system, an ultrasound image view. The method includes automatically detecting, by at least one processor of the ultrasound system, a target view from a set of target views. The target view corresponds with the ultrasound image view. The method includes automatically determining, by the at least one processor, one or both of a presence or absence of a plurality of anatomical features associated with the target view in the ultrasound image view. The method includes presenting, by the at least one processor, at least one user interface element indicating the one or both of the presence or absence of each of the plurality of anatomical features at a display system.

### FIELD

Certain embodiments relate to ultrasound imaging. More specifically, certain embodiments relate to a method and system for adapting user interface elements based on real-time anatomical structure recognition in acquired ultrasound image views. The adapted user interface elements may be configured to indicate protocol adherence or non-adherence by identifying anatomical and/or image features associated with a detected target view that are preset and/or absent in the acquired ultrasound image view.

### BACKGROUND

Ultrasound imaging is a medical imaging technique for imaging organs and soft tissues in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a valuable, non-invasive tool for diagnosing various medical conditions. Several ultrasound examination types are performed based on specific examination protocols that correspond to the particular examination type. For example, examination protocols exist for a number of ultrasound examination types, including obstetric fetal examinations, gynecological examinations, cardiac examinations, and the like. The examination protocols may define a number of specific target views and criteria for adherence of the target views based on the presence of certain anatomical features. For example, the protocol for a second trimester obstetric fetal examination may include a number of pre-defined views, such as a head transcerebellar plane view, a profile sagittal plane view, a face coronal plane view, a sagittal spine view, a four chamber heart view, and the like. Each of the pre-defined views may include criteria for being protocol adherent, such as the presence of certain anatomical features, image features, and the like. As an example, a protocol adherent head transcerebellar plane view of a second trimester obstetric fetal examination may include anatomical features, such as a cerebellum, cavum septum pellucidum, cisterna magna, midline falx, and brain symmetry, and image features, such as a particular magnification of the acquired ultrasound image view. However, ultrasound operators may have difficulty ensuring that all protocol views have been acquired and that the acquired ultrasound image views are protocol adherent.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for adapting user interface elements based on real-time anatomical structure recognition in acquired ultrasound image views, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to adapt user interface elements based on real-time anatomical structure recognition in acquired ultrasound image views, in accordance with various embodiments.

FIG. 2 is an exemplary display presenting an acquired ultrasound image view and user interface elements identifying anatomical features present and missing in the acquired ultrasound image view, the user interface elements presented at a side panel of the display, in accordance with various embodiments.

FIG. 3 illustrates exemplary displays presenting an acquired ultrasound image view and user interface elements identifying anatomical features present and missing in the acquired ultrasound image view, the user interface elements presented at a side panel of a main display and at a touchscreen display, in accordance with various embodiments.

FIG. 4 is an exemplary display presenting an acquired ultrasound image view and user interface elements identifying anatomical features present and missing in the acquired ultrasound image view, the user interface elements presented at a floating panel of the display, in accordance with various embodiments.

FIG. 5 illustrates exemplary displays presenting an acquired ultrasound image view and user interface elements identifying anatomical features present and missing in the acquired ultrasound image view, the user interface elements presented at a floating panel of a main display and at a touchscreen display, in accordance with various embodiments.

FIG. 6 is an exemplary display presenting an acquired ultrasound image view and user interface elements identifying anatomical features present and missing in the acquired ultrasound image view, the user interface elements including a pictogram overlaid on the acquired ultrasound image view, in accordance with various embodiments.

FIG. 7 illustrates exemplary displays presenting an acquired ultrasound image view and user interface elements identifying anatomical features present and missing in the acquired ultrasound image view, the user interface elements including a pictogram overlaid on the acquired ultrasound image view presented at a main display and user interface elements presented at a touchscreen display, in accordance with various embodiments.

FIG. 8 illustrates exemplary displays presenting an acquired ultrasound image view and user interface elements identifying anatomical features present and missing in the acquired ultrasound image view, the user interface elements presented at a main display and including a pictogram overlaid on the acquired ultrasound image view presented at a touchscreen display, in accordance with various embodiments.

FIG. 9 is an exemplary display presenting an acquired ultrasound image view and user interface elements identifying anatomical features present and missing in the acquired ultrasound image view, the user interface elements including structural markers overlaid on the acquired ultrasound image view, in accordance with various embodiments.

FIG. 10 illustrates exemplary displays presenting an acquired ultrasound image view and user interface elements identifying anatomical features present and missing in the acquired ultrasound image view, the user interface elements including structural markers overlaid on the acquired ultrasound image view presented at a main display and user interface elements presented at a touchscreen display, in accordance with various embodiments.

FIG. 11 is an exemplary display presenting an acquired ultrasound image view and user interface elements identifying anatomical features present and missing in the acquired ultrasound image view, the user interface elements including a three-dimensional (3D) model having a representation of a location of the acquired ultrasound image view and instructions for manipulating an ultrasound probe to acquire a protocol adherent view, in accordance with various embodiments.

FIG. 12 illustrates exemplary displays presenting an acquired ultrasound image view and user interface elements identifying anatomical features present and missing in the acquired ultrasound image view, the user interface elements including a three-dimensional (3D) model having a representation of a location of the acquired ultrasound image view and instructions for manipulating an ultrasound probe to acquire a protocol adherent view presented at a main display and user interface elements presented at a touchscreen display, in accordance with various embodiments.

FIG. 13 is a flow chart illustrating exemplary steps that may be utilized for adapting user interface elements based on real-time anatomical structure recognition in acquired ultrasound image views, in accordance with exemplary embodiments.

### DETAILED DESCRIPTION

Certain embodiments may be found in a method and system for adapting user interface elements based on real-time anatomical structure recognition in acquired ultrasound image views. Various embodiments have the technical effect of indicating protocol adherence or non-adherence by identify anatomical and/or image features associated with a detected target view that are present and/or absent in an acquired ultrasound image view. Aspects of the present disclosure have the technical effect of providing user feedback for manipulating an ultrasound probe to acquire a protocol adherent ultrasound image view.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

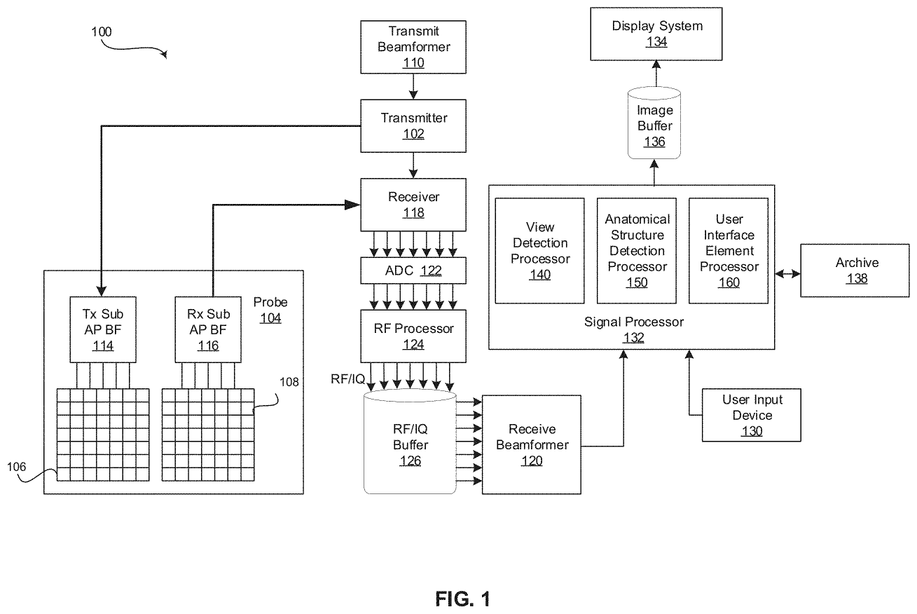

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to adapt user interface elements 220-270 based on real-time anatomical structure recognition in acquired ultrasound image views 210, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select examination types, protocols and/or templates, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, a touch pad, a trackball, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), an enterprise archive (EA), a vendor-neutral archive (VNA), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a view detection processor 140, an anatomical structure detection processor 150, and a user interface element processor 160. The signal processor 132 may be capable of receiving input information from a user input device 130 and/or archive 138, receiving image data, generating an output displayable by a display system 134, and manipulating the output in response to input information from a user input device 130, among other things. The signal processor 132, including the view detection processor 140, the anatomical structure detection processor 150, and the user interface element processor 160, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 frames per second but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a view detection processor 140 that comprises suitable logic, circuitry, interfaces and/or code that may be operable to detect a target view provided by an acquired ultrasound image view. For example, during a second trimester obstetric fetal examination, an associated protocol may define that a number of views be acquired, such as a head transcerebellar plane view, a profile sagittal plane view, a face coronal plane view, a sagittal spine view, a four chamber heart view, and the like. The view detection processor 140 may comprise suitable logic, circuitry, interfaces and/or code that may be operable to provide image analysis techniques to determine which of the target views is provided in the acquired ultrasound image view. In various embodiments, the view detection processor 140 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to detect a view of the acquired ultrasound image view. The artificial intelligence image analysis techniques or machine learning processing functionality configured to provide the view detection functionality may additionally and/or alternatively be provided by a different processor or distributed across multiple processors at the ultrasound system 100 and/or a remote processor communicatively coupled to the ultrasound system 100. For example, the view detection functionality may be provided as a deep neural network that may be made up of, for example, an input layer, an output layer, and one or more hidden layers in between the input and output layers. Each of the layers may be made up of a plurality of processing nodes that may be referred to as neurons. For example, the view detection functionality may include an input layer having a neuron for each pixel or a group of pixels from an acquired ultrasound image view. The output layer may have a neuron corresponding to a plurality of pre-defined ultrasound image target views, such as a head transcerebellar plane view, a profile sagittal plane view, a face coronal plane view, a sagittal spine view, a four chamber heart view, an unknown view, or any suitable target view depending on the examination type. Each neuron of each layer may perform a processing function and pass the processed image information to one of a plurality of neurons of a downstream layer for further processing. As an example, neurons of a first layer may learn to recognize edges of structure in the image data. The neurons of a second layer may learn to recognize shapes based on the detected edges from the first layer. The neurons of a third layer may learn positions of the recognized shapes relative to landmarks in the image data. The processing performed by the deep neural network may identify a target view provided by an acquired ultrasound image view with a high degree of probability.

The signal processor 132 may include an anatomical structure detection processor 150 that comprises suitable logic, circuitry, interfaces and/or code that may be operable to determine whether anatomical and/or image features associated with the detected target view are present or absent in the acquired ultrasound image view. For example, an associated protocol may define that a protocol adherent acquired ultrasound image view of a particular target view, as detected by the view detection processor 140, include particular anatomical and/or image features. As an example, a head transcerebellar plane view of a second trimester obstetric fetal examination may be defined by a protocol as including anatomical features, such as a cerebellum, cavum septum pellucidum, cisterna magna, midline falx, and brain symmetry, and image features, such as a particular magnification of the acquired ultrasound image view. The anatomical structure detection processor 150 may comprise suitable logic, circuitry, interfaces and/or code that may be operable to provide image analysis techniques to determine the features present and absent from the acquired ultrasound image view as defined by the protocol associated with the target view detected by the view detection processor 140. For example, the anatomical structure detection processor 150 may determine spatial probability distributions for a set of anatomical structures defined to be present in a particular view.

In various embodiments, the anatomical structure detection processor 150 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to determine the presence and absence of the features in the acquired ultrasound image view. The artificial intelligence image analysis techniques or machine learning processing functionality configured to provide the feature presence determination functionality may additionally and/or alternatively be provided by a different processor or distributed across multiple processors at the ultrasound system 100 and/or a remote processor communicatively coupled to the ultrasound system 100. For example, the feature presence determination functionality may be provided as a deep neural network that may be made up of, for example, an input layer, an output layer, and one or more hidden layers in between the input and output layers. Each of the layers may be made up of a plurality of processing nodes that may be referred to as neurons. For example, the feature presence determination functionality may include an input layer having a neuron for each pixel or a group of pixels from an acquired ultrasound image view. The output layer may have a neuron corresponding to each combination of present and/or missing features in the acquired ultrasound image view. Each neuron of each layer may perform a processing function and pass the processed image information to one of a plurality of neurons of a downstream layer for further processing. As an example, neurons of a first layer may learn to recognize edges of structure in the image data. The neurons of a second layer may learn to recognize shapes based on the detected edges from the first layer. The neurons of a third layer may learn positions of the recognized shapes relative to landmarks in the image data. The processing performed by the deep neural network may determine the presence and absence of protocol-defined features provided by an acquired ultrasound image view with a high degree of probability.

The signal processor 132 may include a user interface element processor 160 that comprises suitable logic, circuitry, interfaces and/or code that may be operable to generate and present user interface elements 220-270 identifying anatomical structures present and/or absent from the acquired ultrasound image view as determined by the anatomical structure detection processor 150 at the display system 134. For example, the user interface elements 220-270 may comprise pictorial identifiers 220 such as a pictogram 222, 240 and/or structural overlays 250 with markers 224, 226 corresponding with anatomical and/or image features present 224 and/or missing 226 in the acquired ultrasound image view 210 detected as corresponding with a target view. The pictogram 222 may be presented in a side panel, floating panel 200C, and/or separate display 200B from a main display 200A presenting the acquired ultrasound image view 210. In various embodiments, the user interface element processor 160 may be configured to register a pictogram template 240 or structural overlay template 250 with the acquired ultrasound image view 210 and present the pictogram template 240 or structural overlay template 250 overlaid on the acquired ultrasound image view 210 with markers 224, 226 or other identifiers indicating the presence 224 and/or absence 226 of anatomical and/or image features in the acquired ultrasound image view 210.

As another example, the user interface elements 220-270 may comprise a list 230 of anatomical and/or image features present 232 and/or missing 234 in the acquired ultrasound image view 210 detected as corresponding with a target view. The list 230 may be presented in a side panel, center panel, floating panel 200C, and/or separate display 200B from a main display 200A presenting the acquired ultrasound image view 210. As an additional example, the user interface elements may include a three-dimensional (3D) anatomical model 260 having a representation 262 of a location of the acquired ultrasound image view 210. For example, an acquired ultrasound image view 210 of a head transcerebellar plane view of a second trimester obstetric fetal examination may include user interface elements 220-270 comprising a 3D model 260 of a fetus and a plane 262 through the 3D model 260 of the fetus illustrating a current location of the acquired ultrasound image view 210. In yet another exemplary embodiment, the user interface elements 220-270 may additionally and/or alternatively comprise instructions 270 for manipulating an ultrasound probe to acquire a protocol adherent view. For example, the instructions 270 may include text, directional icons, audio, and/or the like providing feedback to an operator for manipulating a position and/or orientation of the ultrasound probe 104 and/or adjusting imaging settings to acquire a protocol adherent view depicting the anatomical and/or image features for a protocol adherent view of the detected target view. The imaging settings may include gain, depth, zoom level, and/or any suitable image setting.

The user interface element processor 160 may comprise suitable logic, circuitry, interfaces and/or code that may be operable to generate and present user interface elements 220-270 at one or more displays 200A, 200B, 200C of the display system 134. Various combinations of user interface elements 220-270 may be displayed at various locations within the displays 200A, 200B, 200C of the display system 134. The generation and presentation of the user interface elements 220-270 by the user interface element processor 160 may be based on an examination type, default settings, user-defined settings, and/or the like.

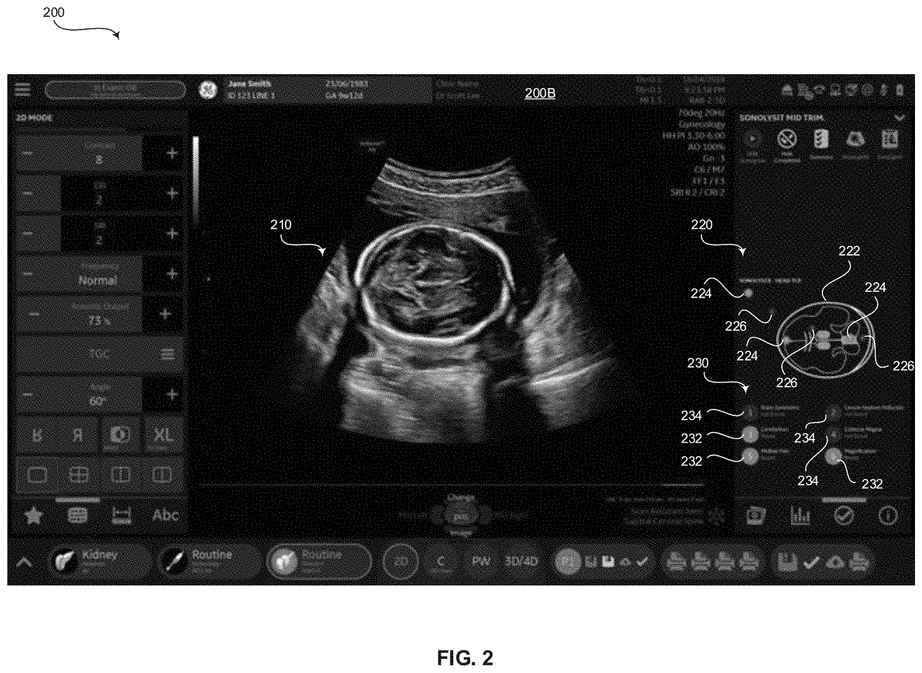

FIG. 2 is an exemplary display 200, 200B presenting an acquired ultrasound image view 210 and user interface elements 220-234 identifying anatomical features present 224, 232 and missing 226, 234 in the acquired ultrasound image view 210, the user interface elements 220-234 presented at a side panel of the display 200, 200B, in accordance with various embodiments. Referring to FIG. 2, the display 200 may be a touchscreen display 200B, a main display, or any suitable display of a display system 134. The display 200 may comprise an acquired ultrasound image view 210 and user interface elements 220-234, among other things. The acquired ultrasound image view 210 may correspond with a target view of a protocol. For example, the acquired ultrasound image view 210 of FIG. 2 illustrates a head transcerebellar plane view of a second trimester obstetric fetal examination. The protocol associated with the head transcerebellar plane view may include anatomical features, such as a cerebellum, cavum septum pellucidum, cisterna magna, midline falx, and brain symmetry, and image features, such as a particular magnification of the acquired ultrasound image view. The user interface elements 220-234 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234 may include pictorial identifiers 220 and a list 230, among other things. The pictorial identifiers 220 may include a pictogram 222 of the anatomical structure and markers identifying the presence 224 and/or absence 226 of anatomical and/or image features. The list 230 may include a list of the anatomical and/or image features present in a protocol adherent view and an indication of whether each of the anatomical and/or image features are present or absent, for example. Still referring to FIG. 2, the markers 224, 226 and list indicators 232, 234 may correspond with, for example, a cerebellum, cavum septum pellucidum, cisterna magna, midline falx, brain symmetry, and a particular magnification of the acquired ultrasound image view 210 as defined by a protocol for a head transcerebellar plane view of a second trimester obstetric fetal examination. The markers 224, 226 and list indicators 232, 234 may distinguish between present and absent features and/or distinguish between different anatomical and/or image features by text, numbering, position, shape, color, or any suitable mechanism. As shown in FIG. 2, the user interface elements 220-234 may be presented at a side panel of the display 200B with the acquired ultrasound image view 210 presented at a center panel of the display 200B.

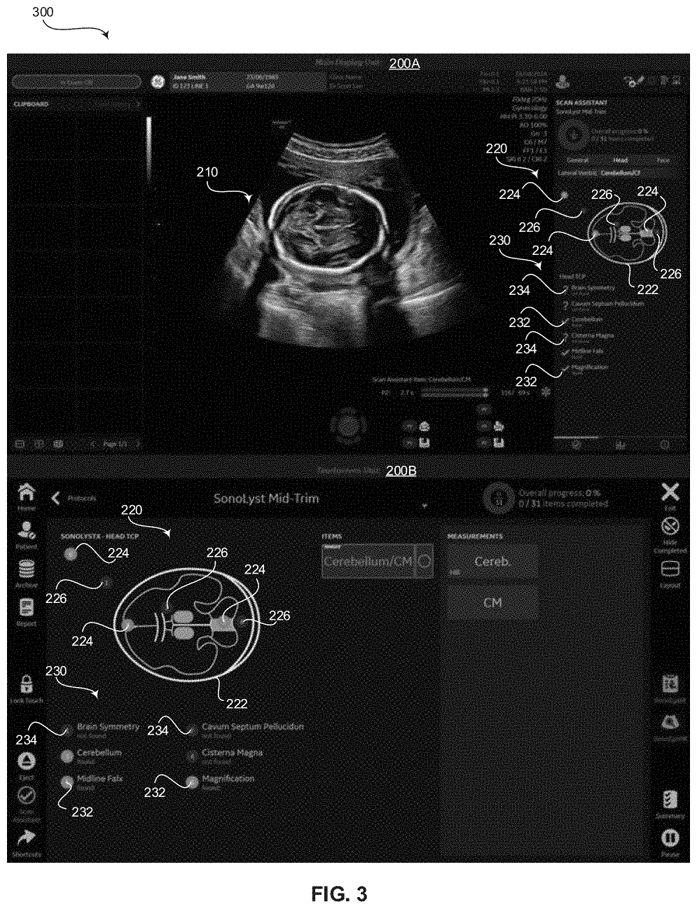

FIG. 3 illustrates exemplary displays 300, 200A, 200B presenting an acquired ultrasound image view 210 and user interface elements 220-234 identifying anatomical features present 224, 232 and missing 226, 234 in the acquired ultrasound image view 210, the user interface elements 220-234 presented at a side panel of a main display 200A and at a touchscreen display 200B, in accordance with various embodiments. Referring to FIG. 3, the display 300 may include a main display 200A, a touchscreen display 200B, and/or any suitable display of a display system 134. The main display 200A of the exemplary embodiment of FIG. 3 may comprise an acquired ultrasound image view 210 and user interface elements 220-234, among other things. The acquired ultrasound image view 210 may correspond with a target view of a protocol. The user interface elements 220-234 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234 may include pictorial identifiers 220 and a list 230, among other things. The pictorial identifiers 220 may include a pictogram 222 of the anatomical structure and markers identifying the presence 224 and/or absence 226 of anatomical and/or image features. The list 230 may include a list of the anatomical and/or image features present in a protocol adherent view and an indication of whether each of the anatomical and/or image features are present or absent, for example. The markers 224, 226 and list indicators 232, 234 may distinguish between present and absent features and/or distinguish between different anatomical and/or image features by text, numbering, position, shape, color, or any suitable mechanism. As shown in FIG. 3, the user interface elements 220-234 may be presented at a side panel of the main display 200A with the acquired ultrasound image view 210 presented at a center panel of the main display 200A. The user interface elements 220-234 may additionally and/or alternatively be presented at the touchscreen display 200B of the display system 134.

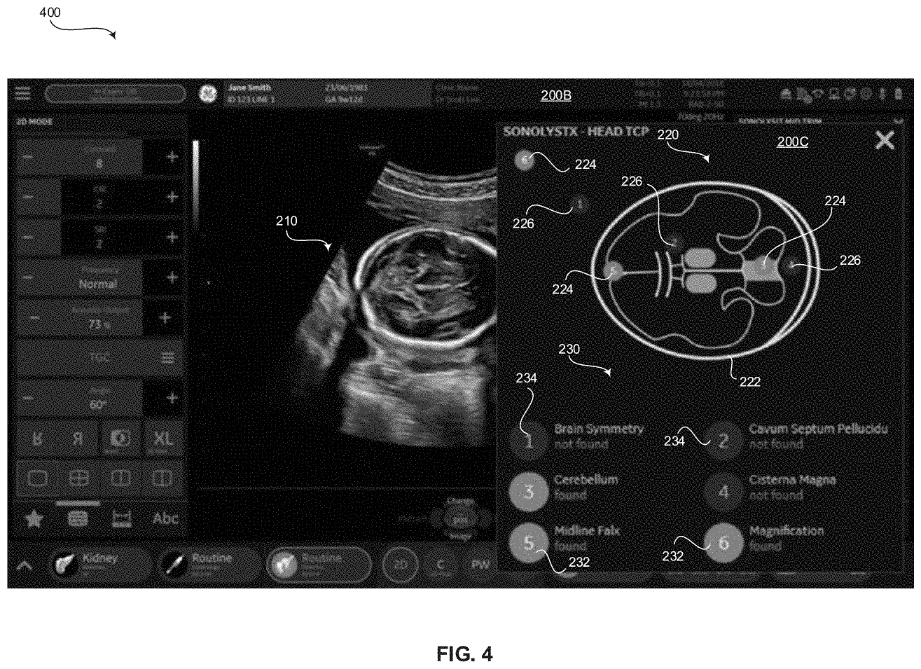

FIG. 4 is an exemplary display 400, 200B, 200C presenting an acquired ultrasound image view 210 and user interface elements 220-234 identifying anatomical features present 224, 232 and missing 226, 234 in the acquired ultrasound image view 210, the user interface elements 220-234 presented at a floating panel 200C of the display 400, 200B, 200C, in accordance with various embodiments. Referring to FIG. 4, the display 400 may be a touchscreen display 200B, a floating display 200C, a main display, or any suitable display of a display system 134. The display 400 may comprise an acquired ultrasound image view 210 presented at the touchscreen display 200B and user interface elements 220-234 presented at the floating display 200C within the touchscreen display 200B, for example. The acquired ultrasound image view 210 may correspond with a target view of a protocol. The user interface elements 220-234 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234 may include pictorial identifiers 220 and a list 230, among other things. The pictorial identifiers 220 may include a pictogram 222 of the anatomical structure and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, or any suitable mechanism. As shown in FIG. 4, the acquired ultrasound image view 210 may be presented at the touchscreen display 200B and the user interface elements 220-234 may be presented in a floating panel 200C within the touchscreen display 200B.

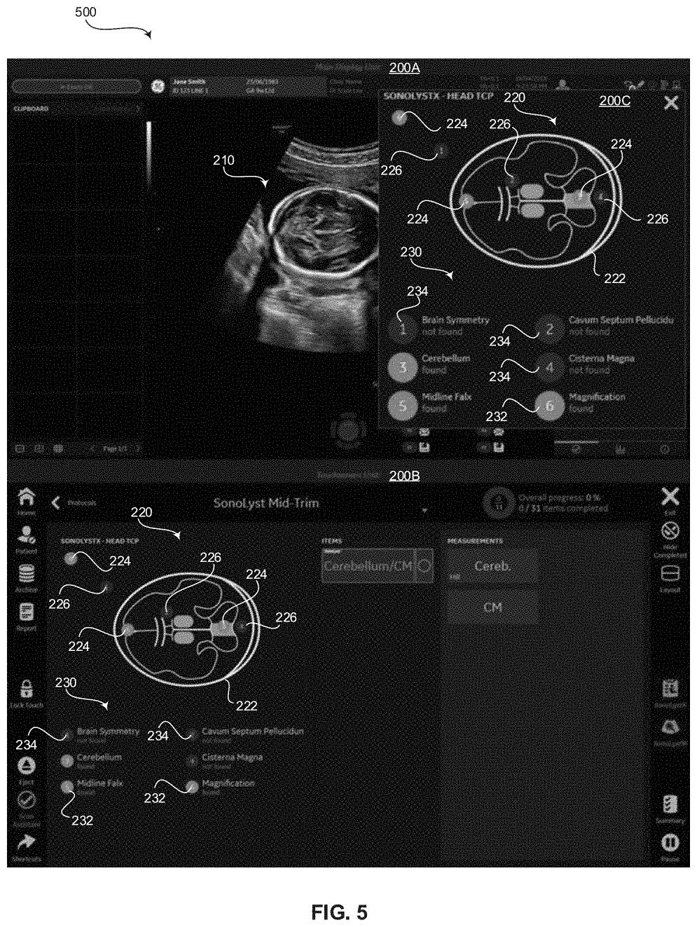

FIG. 5 illustrates exemplary displays 500, 200A, 200B, 200C presenting an acquired ultrasound image view 210 and user interface elements 220-234 identifying anatomical features present 224, 232 and missing 226, 234 in the acquired ultrasound image view 210, the user interface elements 220-234 presented at a floating panel 200C of a main display 200A and at a touchscreen display 200B, in accordance with various embodiments. Referring to FIG. 5, the display 500 may include a main display 200A, a touchscreen display 200B, a floating display 200C within the main display 200A, and/or any suitable display of a display system 134. The main display 200A of the exemplary embodiment of FIG. 5 may comprise an acquired ultrasound image view 210 and the floating display 200C presenting the user interface elements 220-234, among other things. The acquired ultrasound image view 210 may correspond with a target view of a protocol. The user interface elements 220-234 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234 may include pictorial identifiers 220 and a list 230, among other things. The pictorial identifiers 220 may include a pictogram 222 of the anatomical structure and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, and/or any suitable mechanism. As shown in FIG. 5, the user interface elements 220-234 may be presented in a floating display 200C presented within the main display 200A with the acquired ultrasound image view 210 presented in the main display 200A. The user interface elements 220-234 may additionally and/or alternatively be presented at the touchscreen display 200B of the display system 134.

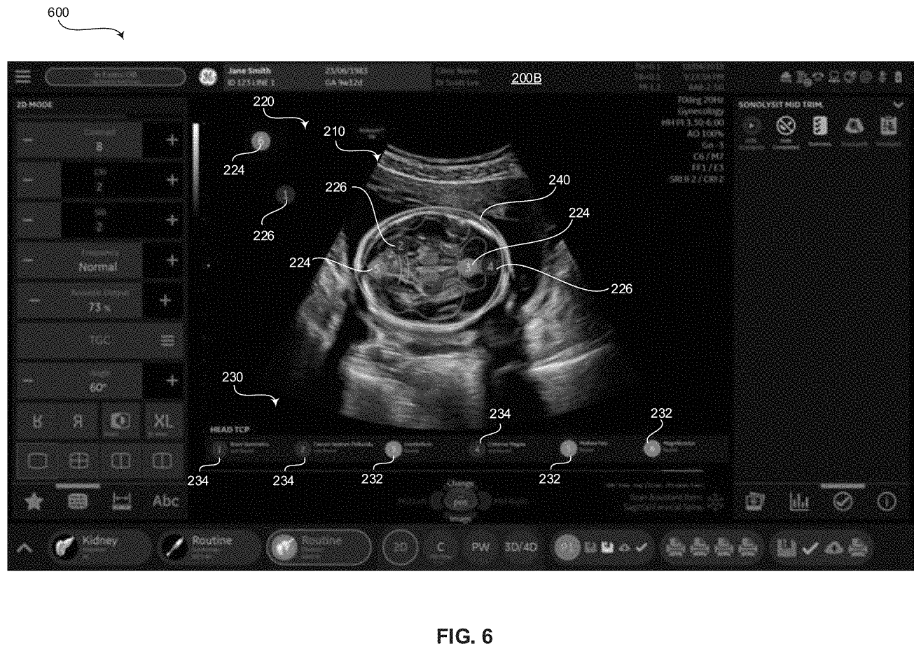

FIG. 6 is an exemplary display 600, 200B presenting an acquired ultrasound image view 210 and user interface elements 220-240 identifying anatomical features present 224, 232 and missing 226, 234 in the acquired ultrasound image view 210, the user interface elements 220-240 including a pictogram 240 overlaid on the acquired ultrasound image view 210, in accordance with various embodiments. Referring to FIG. 6, the display 600 may be a touchscreen display 200B, a main display, or any suitable display of a display system 134. The display 600 may comprise an acquired ultrasound image view 210 and user interface elements 220-240, among other things. The acquired ultrasound image view 210 may correspond with a target view of a protocol. The user interface elements 220-240 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-240 may include pictorial identifiers 220 and a list 230, among other things. The pictorial identifiers 220 may include a pictogram 240 of the anatomical structure registered to and overlaid on the acquired ultrasound image view 210 and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, or any suitable mechanism. As shown in FIG. 6, the user interface elements 220-240 may be presented at the touchscreen display 200B with the acquired ultrasound image view 210.

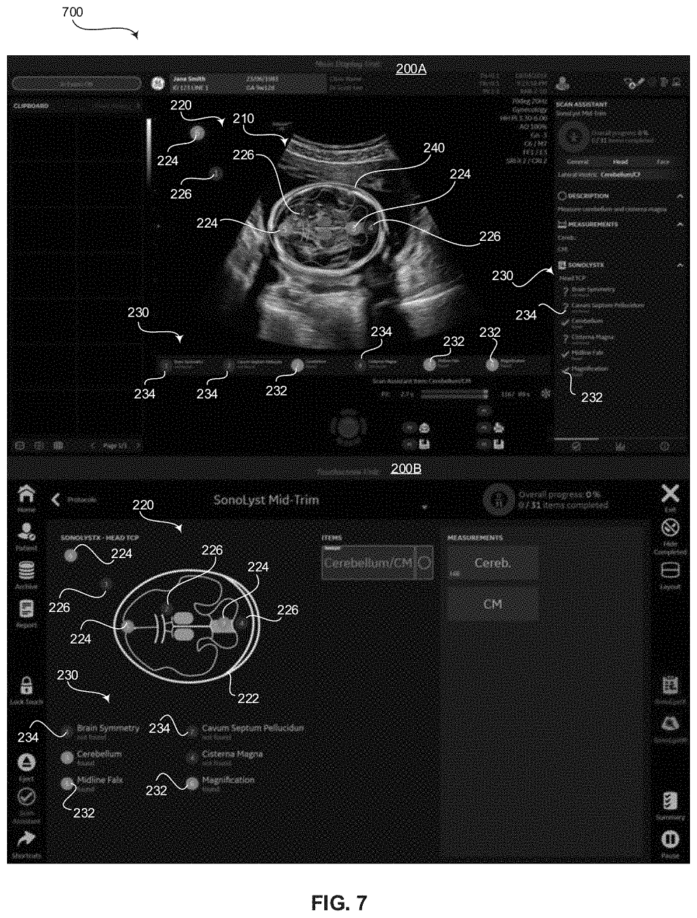

FIG. 7 illustrates exemplary displays 700, 200A, 200B presenting an acquired ultrasound image view 210 and user interface elements 220-240 identifying anatomical features present 224, 232 and missing 226, 234 in the acquired ultrasound image view 210, the user interface elements 220-240 including a pictogram 240 overlaid on the acquired ultrasound image view 210 presented at a main display 200A and user interface elements 220-234 presented at a touchscreen display 200B, in accordance with various embodiments. Referring to FIG. 7, the display 700 may include a main display 200A, a touchscreen display 200B, and/or any suitable display of a display system 134. The main display 200A of the exemplary embodiment of FIG. 7 may comprise an acquired ultrasound image view 210 and the user interface elements 220-240, among other things. The acquired ultrasound image view 210 may correspond with a target view of a protocol. The user interface elements 220-240 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-240 may include pictorial identifiers 220 and a list 230, among other things. The pictorial identifiers 220 may include a pictogram 240 of the anatomical structure registered to and overlaid on the acquired ultrasound image view 210 and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, and/or any suitable mechanism. As shown in FIG. 7, the user interface elements 220-240 may be presented at the main display 200A with the acquired ultrasound image view 210. The user interface elements 220-234 may additionally and/or alternatively be presented at the touchscreen display 200B of the display system 134.

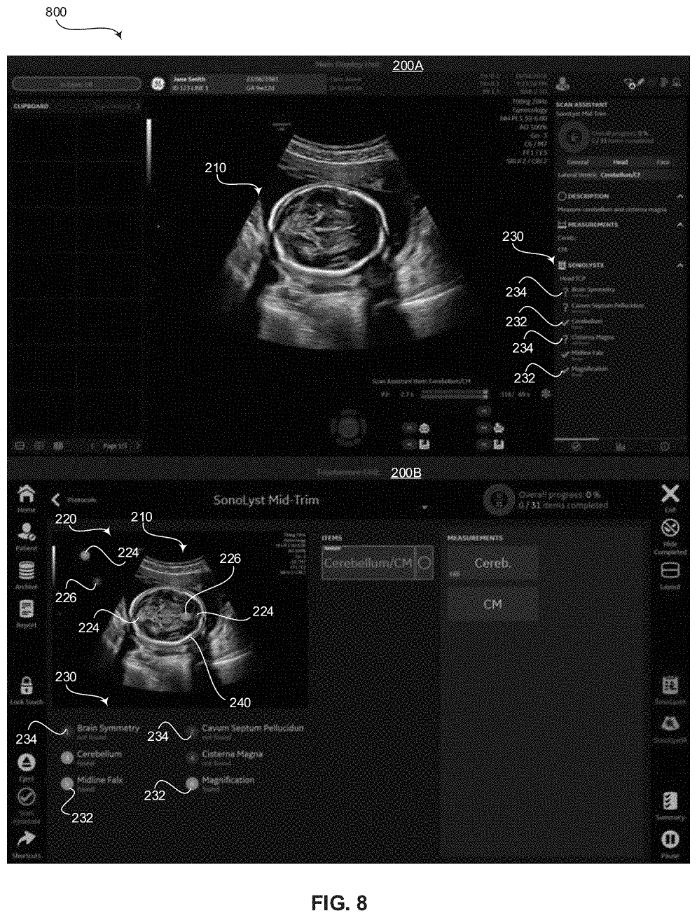

FIG. 8 illustrates exemplary displays 800, 200A, 200B presenting an acquired ultrasound image view 210 and user interface elements 220-240 identifying anatomical features present 224, 232 and missing 226, 234 in the acquired ultrasound image view 210, the user interface elements 230-234 presented at a main display 200A and including a pictogram 240 overlaid on the acquired ultrasound image view 210 presented at a touchscreen display 200B, in accordance with various embodiments. Referring to FIG. 8, the display 800 may include a main display 200A, a touchscreen display 200B, and/or any suitable display of a display system 134. The touchscreen display 200B of the exemplary embodiment of FIG. 8 may comprise an acquired ultrasound image view 210 and the user interface elements 220-240, among other things. The acquired ultrasound image view 210 may correspond with a target view of a protocol. The user interface elements 220-240 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-240 may include pictorial identifiers 220 and a list 230, among other things. The pictorial identifiers 220 may include a pictogram 240 of the anatomical structure registered to and overlaid on the acquired ultrasound image view 210 and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, and/or any suitable mechanism. As shown in FIG. 8, the user interface elements 220-240 may be presented at the touchscreen display 200B with the acquired ultrasound image view 210. The user interface elements 220-234 and acquired ultrasound image view 210 may additionally and/or alternatively be presented at the main display 200A of the display system 134.

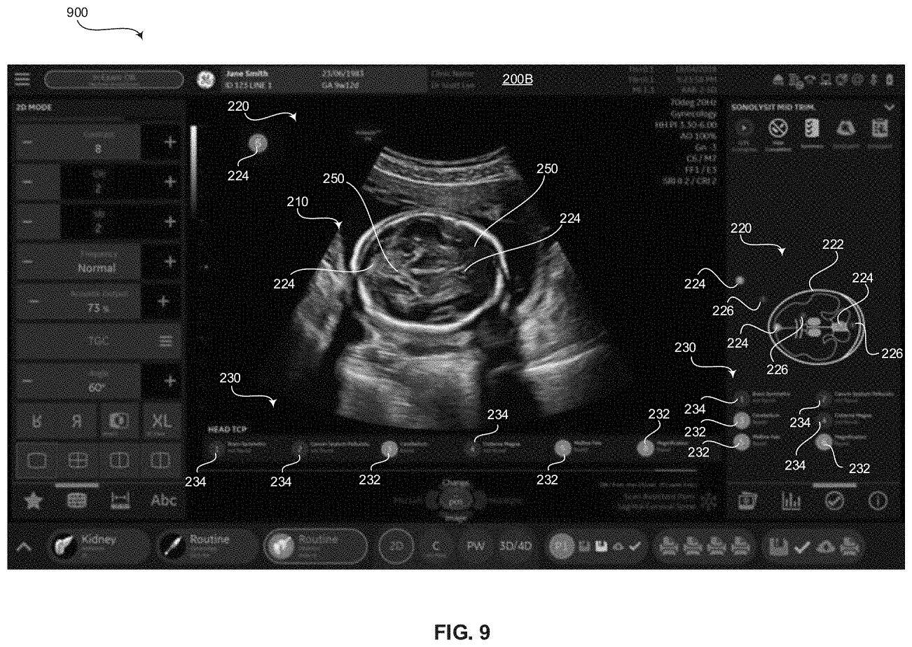

FIG. 9 is an exemplary display 900, 200B presenting an acquired ultrasound image view 210 and user interface elements 220-234, 250 identifying anatomical features present 224, 232 and missing 226, 234 in the acquired ultrasound image view 210, the user interface elements 220-234, 250 including structural markers 250 overlaid on the acquired ultrasound image view 210, in accordance with various embodiments. Referring to FIG. 9, the display 900 may be a touchscreen display 200B, a main display, or any suitable display of a display system 134. The display 900 may comprise an acquired ultrasound image view 210 and user interface elements 220-234, 250, among other things. The acquired ultrasound image view 210 may correspond with a target view of a protocol. The user interface elements 220-234, 250 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234, 250 may include pictorial identifiers 220 and a list 230, among other things. The pictorial identifiers 220 may include structural markers 250 (also referred to as overlaid anatomical structures 250) registered to and superimposed on the acquired ultrasound image view 210 and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, or any suitable mechanism. As shown in FIG. 9, the user interface elements 220-234, 250 may be presented at the touchscreen display 200B with the acquired ultrasound image view 210.

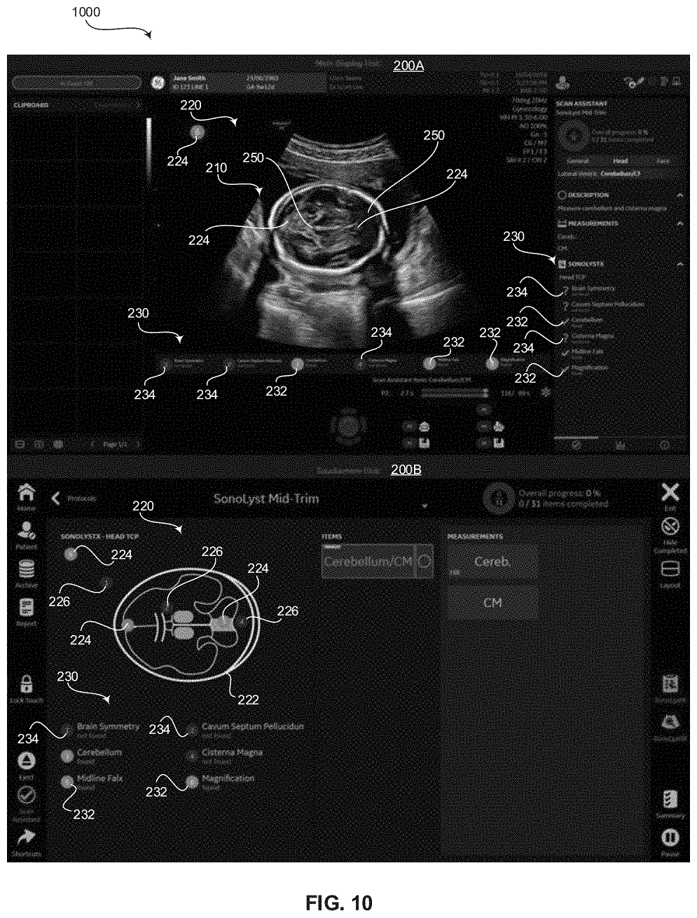

FIG. 10 illustrates exemplary displays 1000, 200A, 200B presenting an acquired ultrasound image view 210 and user interface elements 220-234, 250 identifying anatomical features present 224, 232 and missing 226, 234 in the acquired ultrasound image view 210, the user interface elements 220-234, 250 including structural markers 250 overlaid on the acquired ultrasound image view 210 presented at a main display 200A and user interface elements 220-234 presented at a touchscreen display 200B, in accordance with various embodiments. Referring to FIG. 10, the display 1000 may include a main display 200A, a touchscreen display 200B, and/or any suitable display of a display system 134. The main display 200A of the exemplary embodiment of FIG. 10 may comprise an acquired ultrasound image view 210 and the user interface elements 220-234, 250, among other things. The acquired ultrasound image view 210 may correspond with a target view of a protocol. The user interface elements 220-234, 250 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234, 250 may include pictorial identifiers 220 and a list 230, among other things. The pictorial identifiers 220 may include structural markers 250 (also referred to as overlaid anatomical structures 250) registered to and superimposed on the acquired ultrasound image view 210 and markers identifying the presence 224 and/or absence 226 of anatomical and/or image features. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, and/or any suitable mechanism. As shown in FIG. 10, the user interface elements 220-234, 250 may be presented at the main display 200A with the acquired ultrasound image view 210. The user interface elements 220-234 may additionally and/or alternatively be presented at the touchscreen display 200B of the display system 134.

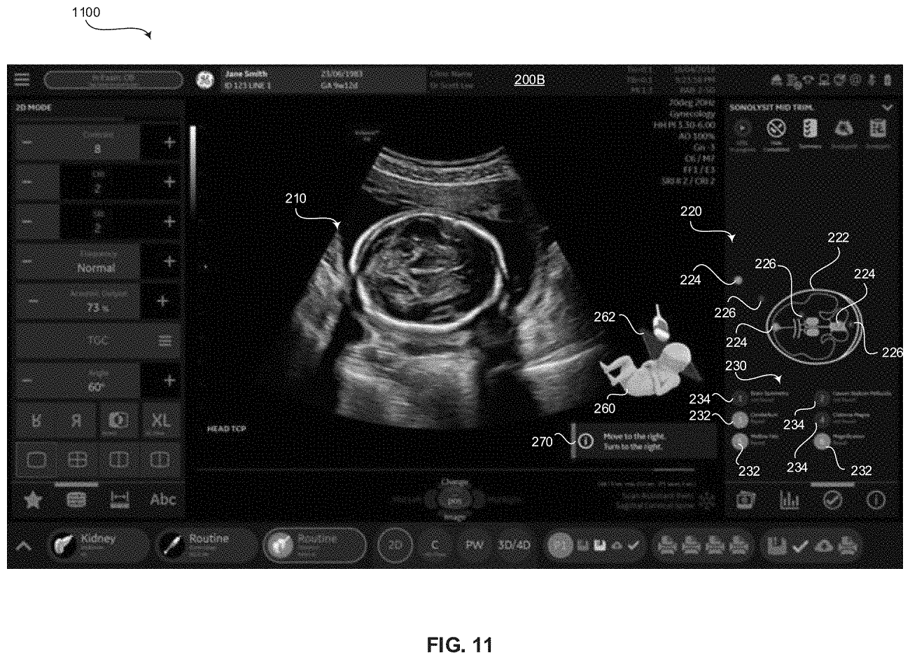

FIG. 11 is an exemplary display 1100, 200B presenting an acquired ultrasound image view 210 and user interface elements 220-234, 260-270 identifying anatomical features present 224, 232 and missing 226, 234 in the acquired ultrasound image view 210, the user interface elements 220-234, 260-270 including a three-dimensional (3D) model 260 having a representation 262 of a location of the acquired ultrasound image view 210 and instructions 270 for manipulating an ultrasound probe 104 to acquire a protocol adherent view, in accordance with various embodiments. Referring to FIG. 11, the display 1100 may be a touchscreen display 200B, a main display, or any suitable display of a display system 134. The display 1100 may comprise an acquired ultrasound image view 210 and user interface elements 220-234, 260-270, among other things. The acquired ultrasound image view 210 may correspond with a target view of a protocol. The user interface elements 220-234, 260-270 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234, 260-270 may include pictorial identifiers 220 and a list 230, among other things. The pictorial identifiers 220 may include a 3D model 260 of the anatomy, a representation 262 of plane with respect to the 3D model corresponding with the acquired ultrasound image view 210, instructions 270 for manipulating an ultrasound probe 104 to acquire a protocol adherent view, a pictogram 222, and/or markers identifying the presence 224 and/or absence 226 of anatomical and/or image features. The instructions 270 may include text, directional icons, and/or any suitable instructions for adjusting probe settings and/or manipulating a probe position and/or orientation to acquire an ultrasound image view 210 having the anatomical and/or image features for a detected view as defined by the protocol. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, or any suitable mechanism. As shown in FIG. 11, the user interface elements 220-234, 260-270 may be presented at the touchscreen display 200B with the acquired ultrasound image view 210.

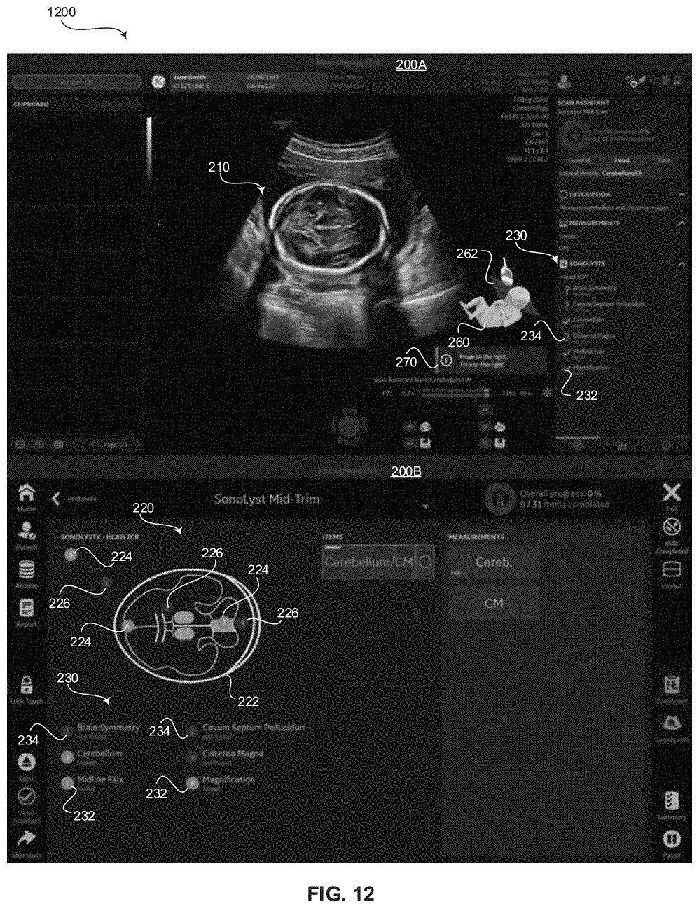

FIG. 12 illustrates exemplary displays 1200, 200A, 200B presenting an acquired ultrasound image view 210 and user interface elements 220-234, 260-270 identifying anatomical features present 224, 232 and missing 226, 234 in the acquired ultrasound image view 210, the user interface elements 220-234, 260-270 including a three-dimensional (3D) model 360 having a representation 362 of a location of the acquired ultrasound image view and instructions 270 for manipulating an ultrasound probe 104 to acquire a protocol adherent view presented at a main display 200A and user interface elements 220-234 presented at a touchscreen display 200B, in accordance with various embodiments. Referring to FIG. 12, the display 1200 may include a main display 200A, a touchscreen display 200B, and/or any suitable display of a display system 134. The main display 200A of the exemplary embodiment of FIG. 12 may comprise an acquired ultrasound image view 210 and the user interface elements 220-234, 260-270, among other things. The acquired ultrasound image view 210 may correspond with a target view of a protocol. The user interface elements 220-234, 260-270 provide feedback regarding whether the acquired ultrasound image view 210 is a protocol adherent view by identifying the anatomical and/or image features that are present 224, 232 and/or missing 226, 234 in the acquired ultrasound image view 210. The user interface elements 220-234, 260-270 may include pictorial identifiers 220 and a list 230, among other things. The pictorial identifiers 220 may include a 3D model 260 of the anatomy, a representation 262 of plane with respect to the 3D model corresponding with the acquired ultrasound image view 210, instructions 270 for manipulating an ultrasound probe 104 to acquire a protocol adherent view, a pictogram 222, and/or markers identifying the presence 224 and/or absence 226 of anatomical and/or image features. The instructions 270 may include text, directional icons, and/or any suitable instructions for adjusting probe settings and/or manipulating a probe position and/or orientation to acquire an ultrasound image view 210 having the anatomical and/or image features for a detected view as defined by the protocol. The list 230 may include a list of the anatomical and/or image features present in a protocol adherent view and an indication of whether each of the anatomical and/or image features are present 232 or absent 234, for example. The markers 224, 226 and list indicators 232, 234 may distinguish between present and absent features and/or distinguish between different anatomical and/or image features by text, numbering, position, shape, color, and/or any suitable mechanism. As shown in FIG. 12, the user interface elements 220, 230-234, 260-270 may be presented at the main display 200A with the acquired ultrasound image view 210. The user interface elements 220-234 may additionally and/or alternatively be presented at the touchscreen display 200B of the display system 134.

Referring again to FIG. 1, the display system 134 may be any device(s) capable of communicating visual information to a user. For example, a display system 134 may include a liquid crystal display, a light emitting diode display, and/or any suitable display or displays. The display system 134 can be operable to display information from the signal processor 132 and/or archive 138, such as acquired ultrasound image views 210, user interface elements 220-270, such as pictograms 222, markers 224, 226, list items 230, 232, 234, overlaid pictograms 240, overlaid anatomical structures 250 (also referred to as structural markers 250), 3D anatomical models 260, a spatial representation of a current image plane 262, instructions 270 for manipulating an ultrasound probe 104, and/or any suitable information. In various embodiments, one or more of the displays of the display system 134 may be and/or include a touchscreen display 200B.

The archive 138 may be one or more computer-readable memories integrated with the ultrasound system 100 and/or communicatively coupled (e.g., over a network) to the ultrasound system 100, such as a Picture Archiving and Communication System (PACS), an enterprise archive (EA), a vendor-neutral archive (VNA), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores acquired ultrasound image views 210, instructions for detecting a view of the acquired ultrasound image views 210, instructions for detecting anatomical and/or image features in the acquired ultrasound image views 210, and/or instructions for presenting user interface elements 220-270 based on a detected target view and detected features associated with the detected target view that are present and/or absent from the acquired ultrasound image view 210, among other things.

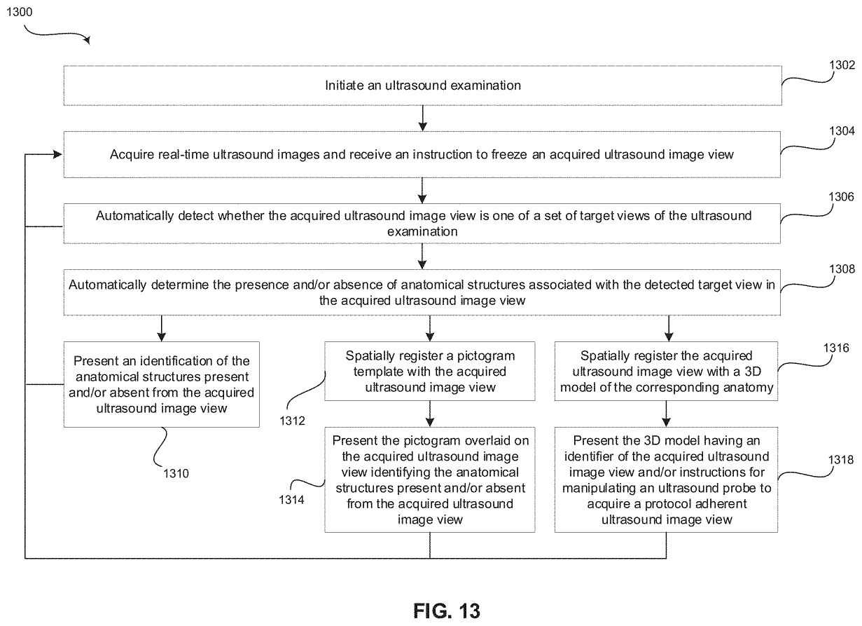

FIG. 13 is a flow chart 1300 illustrating exemplary steps 1302-1318 that may be utilized for adapting user interface elements 220-270 based on real-time anatomical structure recognition in acquired ultrasound image views 210, in accordance with exemplary embodiments. Referring to FIG. 13, there is shown a flow chart 1300 comprising exemplary steps 1302 through 1318. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 1302, an ultrasound examination may be initiated at an ultrasound system 100. For example, an operator of an ultrasound system 100 may select, via a user input device 130, an examination type, such as an obstetric fetal examination, a gynecological examination, a cardiac examination, or the like. The selected examination type may be associated with an examination protocol defining a number of specific target views and criteria for adherence of the target views based on the presence of certain anatomical features. For example, the protocol for a second trimester obstetric fetal examination may include a number of pre-defined views, such as a head transcerebellar plane view, a profile sagittal plane view, a face coronal plane view, a sagittal spine view, a four chamber heart view, and the like. Each of the pre-defined views may include criteria for being protocol adherent, such as the presence of certain anatomical features, image features, and the like. As an example, a protocol adherent head transcerebellar plane view of a second trimester obstetric fetal examination may include anatomical features, such as a cerebellum, cavum septum pellucidum, cisterna magna, midline falx, and brain symmetry, and image features, such as a particular magnification of the acquired ultrasound image view.

At step 1304, the ultrasound system 100 may acquire a real-time ultrasound images and receive an instruction to freeze an acquired ultrasound image view 210. For example, an ultrasound probe 104 of the ultrasound system 100 may acquire real-time ultrasound images of an anatomical structure. A signal processor 132 may receive an instruction from a user input device 130 for freezing an acquired ultrasound image view 210.

At step 1306, the signal processor 132 of the ultrasound system 100 may automatically detect whether the acquired ultrasound image view 210 is one of a set of target view of the ultrasound examination. For example, the view detection processor 140 of the signal processor 132 may be configured to detect a target view provided by an acquired ultrasound image view 210. As an example, if a second trimester obstetric fetal examination is selected at step 1302, an associated protocol may define that a number of views be acquired, such as a head transcerebellar plane view, a profile sagittal plane view, a face coronal plane view, a sagittal spine view, a four chamber heart view, and the like. The view detection processor 140 may be operable to provide image analysis techniques, such as artificial intelligence image analysis algorithms, one or more deep neural networks (e.g., a convolutional neural network such as u-net) and/or any suitable form of artificial intelligence image analysis techniques or machine learning processing functionality, to determine which of the target views is provided in the acquired ultrasound image view 210 acquired at step 1304. In various embodiments, if the detected view is an unknown view or otherwise not one of the target views, the process 1300 may return to step 1304 to acquire a different ultrasound image view 210.

At step 1308, the signal processor 132 of the ultrasound system 100 may automatically determine a presence and/or absence of anatomical structures associated with the detected target view in the acquired ultrasound image view 210. For example, an anatomical structure detection processor 150 of the signal processor 132 may be configured to determine whether anatomical and/or image features associated with the detected target view are present or absent in the acquired ultrasound image view 210. The protocol associated with the examination type selected at step 1302 may define that a protocol adherent acquired ultrasound image view 210 of a particular target view, as detected by the view detection processor 140 at step 1306, include particular anatomical and/or image features. As an example, a head transcerebellar plane view of a second trimester obstetric fetal examination may be defined by a protocol as including anatomical features, such as a cerebellum, cavum septum pellucidum, cisterna magna, midline falx, and brain symmetry, and image features, such as a particular magnification of the acquired ultrasound image view. The anatomical structure detection processor 150 may be operable to provide image analysis techniques, such as artificial intelligence image analysis algorithms, one or more deep neural networks (e.g., a convolutional neural network such as u-net) and/or any suitable form of artificial intelligence image analysis techniques or machine learning processing functionality, to determine the features present and absent from the acquired ultrasound image view 210 as defined by the protocol associated with the target view detected by the view detection processor 140 at step 1306. In various embodiments, the anatomical structure detection processor 150 may determine spatial probability distributions for a set of anatomical structures defined to be present in a particular view.

At step 1310, the signal processor 132 of the ultrasound system 100 may present an identification 220-234 of the anatomical structures present 224, 232 and/or absent 226, 234 from the acquired ultrasound image view 210. For example, a user interface element processor 160 of the signal processor 132 may be configured to generate and present user interface elements 220-234 identifying anatomical structures present 224, 232 and/or absent 226, 234 from the acquired ultrasound image view 210 as determined by the anatomical structure detection processor 150 at the display system 134 at step 1308. The user interface elements 220-234 may comprise pictorial identifiers 220 such as a pictogram 222 with markers 224, 226 corresponding with anatomical and/or image features present 224 and/or missing 226 in the acquired ultrasound image view 210 detected, at step 1306, as corresponding with a target view. The pictogram 222 may be presented in a side panel, floating panel 200C, and/or separate display 200B from a main display 200A presenting the acquired ultrasound image view 210 as illustrated, for example, in FIGS. 2-5 and described above. As another example, the user interface elements 220-234 may comprise a list 230 of anatomical and/or image features present 232 and/or missing 234 in the acquired ultrasound image view 210 detected as corresponding with a target view at step 1306. The list 230 may be presented in a side panel, center panel, floating panel 200C, and/or separate display 200B from a main display 200A presenting the acquired ultrasound image view 210. In various embodiments, if the acquired ultrasound image view 210 is not protocol adherent (e.g., is missing anatomical and/or image features), the process 1300 may return to step 1304 to acquire a different ultrasound image view 210.

At step 1312, the signal processor 132 of the ultrasound system 100 may spatially register a pictogram template 240 with the acquired ultrasound image view 210. For example, the user interface element processor 160 may be configured to register the pictogram template 240 or a structural overlay template 250 with the acquired ultrasound image view 210. The pictogram template 240 and/or structural overlay template 250 may be associated with a particular target view and correspond with the anatomical structure(s) depicted in the acquired ultrasound image view 210.

At step 1314, the signal processor 132 of the ultrasound system 100 may present the pictogram 240 overlaid on the acquired ultrasound image view 210 identifying the anatomical structures present 224, 232 and/or absent 226, 234 from the acquired ultrasound image view 210. For example, the user interface element processor 160 of the signal processor 132 may be configured to present pictorial identifiers 220 such as a pictogram 240 and/or structural overlays 250 with markers 224, 226 corresponding with anatomical and/or image features present 224 and/or missing 226 in the acquired ultrasound image view 210 detected, at step 1306, as corresponding with a target view as shown, for example, in FIGS. 6-10 and as described above. In various embodiments, if the acquired ultrasound image view 210 is not protocol adherent (e.g., is missing anatomical and/or image features), the process 1300 may return to step 1304 to acquire a different ultrasound image view 210.

At step 1316, the signal processor 132 of the ultrasound system 100 may spatially register the acquired ultrasound image view 210 with a 3D model 260 of the corresponding anatomy. For example, the user interface element processor 160 of the signal processor 132 may be configured to register an acquired ultrasound image view 210 of a fetal anatomy with a 3D model 260 of a fetus.

At step 1318, the signal processor 132 of the ultrasound system 100 may present the 3D model 260 having an identifier of the acquired ultrasound image view 210 and/or instructions 270 for manipulating an ultrasound probe 104 to acquire a protocol adherent ultrasound image view. For example, the user interface element processor 160 of the signal processor 132 may be configured to present the 3D model 260 generated at step 1316 with a representation 262 of the location of the acquired ultrasound image view 210 at a display system 134. As another example, the user interface element processor 160 may be configured to generate and present instructions 270 for manipulating an ultrasound probe 104 to acquire a protocol adherent view. For example, the instructions 270 may include text, directional icons, audio, and/or the like providing feedback to an operator for manipulating a position and/or orientation of the ultrasound probe 104 and/or adjusting imaging settings to acquire a protocol adherent view depicting the anatomical and/or image features for a protocol adherent view of the detected target view. The imaging settings may include gain, depth, zoom level, and/or any suitable image setting.

The 3D model 260, plane representation 262, and/or instructions 270 may be presented at a display 1100, 1200, 200A of a display system 134 as illustrated, for example, in FIGS. 11-12 and as described above. In various embodiments, the process 1300 may return to step 1304 to acquire a different ultrasound image view 210 pursuant to the instructions 270.

Aspects of the present disclosure provide a system 100 and method 1300 for adapting user interface elements 220-270 based on real-time anatomical structure recognition in acquired ultrasound image views 210. In accordance with various embodiments, the method 1300 may comprise acquiring 1304, by an ultrasound system 100, an ultrasound image view 210. The method 1300 may comprise automatically detecting 1306, by at least one processor 132, 140 of the ultrasound system 100, a target view from a set of target views. The target view corresponds with the ultrasound image view 210. The method 1300 may comprise automatically determining 1308, by the at least one processor 132, 150, one or both of a presence or absence of a plurality of anatomical features associated with the target view in the ultrasound image view 210. The method 1300 may comprise presenting 1310, 1314, 1318, by the at least one processor 132, 160, at least one user interface element 220-270 indicating the one or both of the presence 224, 232 or absence 226, 234 of each of the plurality of anatomical features at a display system 134.

In a representative embodiment, the method 1300 may comprise receiving 1302, by the at least one processor 132, a selection of an examination type associated with the set of target views. In an exemplary embodiment, the at least one user interface element 220-270 comprises a pictogram 222, 240 of anatomy of the target view. The pictogram 222, 240 may comprise markers 224, 226, indicating the one or both of the presence 224 or absence 226 of each of the plurality of anatomical features. In various embodiments, the method 1300 may comprise registering 1312, by the at least one processor 132, 160, the pictogram 240 to the ultrasound image view 210. The method 1300 may comprise overlaying 1314, by the at least one processor 132, 160, the pictogram 240 on the ultrasound image view 210. In certain embodiments, the at least one user interface element 220-270 comprises a list 230 indicating the one or both of the presence 232 or absence 234 of each of the plurality of anatomical features. In a representative embodiment, the at least one user interface element 220-270 comprises a three-dimensional (3D) model 260 of an anatomy having a representation 262 of a location of the ultrasound image view 210. In an exemplary embodiment, the at least one user interface element 220-270 comprises instructions for one or both of adjusting imaging settings or manipulating one or both of a position and orientation of an ultrasound probe 104 of the ultrasound system 100.

Various embodiments provide an ultrasound system 100 for adapting user interface elements 220-270 based on real-time anatomical structure recognition in acquired ultrasound image views 210. The ultrasound system 100 may comprise an ultrasound probe 104, at least one processor 132, 140, 150, 160 and a display system 134. The ultrasound probe 104 may be configured to acquire an ultrasound image view 210. The at least one processor 132, 140 may be configured to automatically detect a target view from a set of target views. The target view corresponds with the ultrasound image view 210. The at least one processor 132, 150 may be configured to automatically determine one or both of a presence or absence of a plurality of anatomical features associated with the target view in the ultrasound image view 210. The at least one processor 132, 160 may be configured to generate at least one user interface element 220-270 indicating the one or both of the presence 224, 232 or absence 226, 234 of each of the plurality of anatomical features. The display system may be configured to present the at least one user interface element 220-270 and the ultrasound image view 210.

In an exemplary embodiment, the ultrasound system 100 comprises a user input device 130 configured to provide the at least one processor 132 with a selection of an examination type associated with the set of target views. In various embodiments, the at least one user interface element 220-270 comprises a pictogram 222, 240 of anatomy of the target view. The at least one processor 132, 160 may be configured to superimpose markers 224, 226 indicating the one or both of the presence 224 or absence 226 of each of the plurality of anatomical features on the pictogram 222, 240. In certain embodiments, the at least one processor 132, 160 may be configured to register the pictogram 240 to the ultrasound image view 210 and superimpose the pictogram 240 on the ultrasound image view 210. In a representative embodiment, the at least one user interface element 220-270 comprises a list 230 indicating 232, 234 the one or both of the presence 232 or absence 234 of each of the plurality of anatomical features. In an exemplary embodiment, the at least one user interface element 220-270 comprises a three-dimensional (3D) model 260 of an anatomy having a representation 262 of a location of the ultrasound image view 210. In various embodiments, the at least one user interface element 220-270 comprises instructions 270 for one or both of adjusting imaging settings or manipulating one or both of a position and orientation of the ultrasound probe 104 of the ultrasound system 100.

Certain embodiments provide a non-transitory computer readable medium having stored thereon, a computer program having at least one code section. The at least one code section is executable by a machine for causing an ultrasound system 100 to perform steps 1300. The steps 1300 may comprise acquiring 1304 an ultrasound image view 210. The steps 1300 may comprise automatically detecting 1306 a target view from a set of target views. The target view corresponds with the ultrasound image view 210. The steps 1300 may comprise automatically determining 1308 one or both of a presence or absence of a plurality of anatomical features associated with the target view in the ultrasound image view 210. The steps 1300 may comprise presenting 1310, 1314, 1318 at least one user interface element 220-270 indicating the one or both of the presence 224, 232 or absence 226, 234 of each of the plurality of anatomical features at a display system 134.

In various embodiments, the at least one user interface element 220-270 comprises a pictogram 222, 240 of anatomy of the target view. The pictogram 222, 240 may comprise markers 224, 226 indicating the one or both of the presence 224 or absence 226 of each of the plurality of anatomical features. In certain embodiments, the steps 1300 may comprise registering 1312 the pictogram 240 to the ultrasound image view 210. The steps 1300 may comprise overlaying 1314 the pictogram 240 on the ultrasound image view 210. In a representative embodiment, the at least one user interface element 220-270 comprises a list 230 indicating 232, 234 the one or both of the presence 232 or absence 234 of each of the plurality of anatomical features. In an exemplary embodiment, the at least one user interface element 220-270 comprises a three-dimensional (3D) model 260 of an anatomy having a representation 262 of a location of the ultrasound image view 210. In various embodiments, the at least one user interface element 220-270 comprises instructions 270 for one or both of adjusting imaging settings or manipulating one or both of a position and orientation of an ultrasound probe 104 of the ultrasound system 100.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for adapting user interface elements based on real-time anatomical structure recognition in acquired ultrasound image views.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by an ultrasound system, an ultrasound image view;
automatically detecting, by at least one processor of the ultrasound system, a target view from a set of target views, the target view corresponding with the ultrasound image view;
automatically determining, by the at least one processor, one or both of a presence or absence of a plurality of anatomical features associated with the target view in the ultrasound image view; and
presenting, by the at least one processor, at least one user interface element indicating the one or both of the presence or absence of each of the plurality of anatomical features at a display system.

2. The method of claim 1, comprising receiving, by the at least one processor, a selection of an examination type associated with the set of target views.

3. The method of claim 1, wherein the at least one user interface element comprises a pictogram of anatomy of the target view, the pictogram comprising markers indicating the one or both of the presence or absence of each of the plurality of anatomical features.

4. The method of claim 3, comprising:
registering, by the at least one processor, the pictogram to the ultrasound image view, and
overlaying, by the at least one processor, the pictogram on the ultrasound image view.

5. The method of claim 1, wherein the at least one user interface element comprises a list indicating the one or both of the presence or absence of each of the plurality of anatomical features.

6. The method of claim 1, wherein the at least one user interface element comprises a three-dimensional (3D) model of an anatomy having a representation of a location of the ultrasound image view.

7. The method of claim 1, wherein the at least one user interface element comprises instructions for one or both of adjusting imaging settings or manipulating one or both of a position and orientation of an ultrasound probe of the ultrasound system.

8. An ultrasound system comprising:
an ultrasound probe configured to acquire an ultrasound image view;
at least one processor configured to:
automatically detect a target view from a set of target views, the target view corresponding with the ultrasound image view;
automatically determine one or both of a presence or absence of a plurality of anatomical features associated with the target view in the ultrasound image view; and
generate at least one user interface element indicating the one or both of the presence or absence of each of the plurality of anatomical features; and
a display system configured to present the at least one user interface element and the ultrasound image view.

9. The system of claim 8, comprising a user input device configured to provide the at least one processor with a selection of an examination type associated with the set of target views.

10. The system of claim 8, wherein the at least one user interface element comprises a pictogram of anatomy of the target view, and wherein the at least one processor is configure to superimpose markers indicating the one or both of the presence or absence of each of the plurality of anatomical features on the pictogram.

11. The system of claim 8, wherein the at least one processor is configured to register the pictogram to the ultrasound image view and superimpose the pictogram on the ultrasound image view.

12. The system of claim 8, wherein the at least one user interface element comprises a list indicating the one or both of the presence or absence of each of the plurality of anatomical features.

13. The system of claim 8, wherein the at least one user interface element comprises a three-dimensional (3D) model of an anatomy having a representation of a location of the ultrasound image view.

14. The system of claim 8, wherein the at least one user interface element comprises instructions for one or both of adjusting imaging settings or manipulating one or both of a position and orientation of the ultrasound probe of the ultrasound system.

15. A non-transitory computer readable medium having stored thereon, a computer program having at least one code section, the at least one code section being executable by a machine for causing an ultrasound system to perform steps comprising:
acquiring an ultrasound image view;
automatically detecting a target view from a set of target views, the target view corresponding with the ultrasound image view;
automatically determining one or both of a presence or absence of a plurality of anatomical features associated with the target view in the ultrasound image view; and
presenting at least one user interface element indicating the one or both of the presence or absence of each of the plurality of anatomical features at a display system.

16. The non-transitory computer readable medium of claim 15, wherein the at least one user interface element comprises a pictogram of anatomy of the target view, the pictogram comprising markers indicating the one or both of the presence or absence of each of the plurality of anatomical features.

17. The non-transitory computer readable medium of claim 16, comprising:
registering the pictogram to the ultrasound image view, and
overlaying the pictogram on the ultrasound image view.

18. The non-transitory computer readable medium of claim 15, wherein the at least one user interface element comprises a list indicating the one or both of the presence or absence of each of the plurality of anatomical features.

19. The non-transitory computer readable medium of claim 15, wherein the at least one user interface element comprises a three-dimensional (3D) model of an anatomy having a representation of a location of the ultrasound image view.

20. The non-transitory computer readable medium of claim 15, wherein the at least one user interface element comprises instructions for one or both of adjusting imaging settings or manipulating one or both of a position and orientation of an ultrasound probe of the ultrasound system.
